(19) United States
(12) Patent Application Publication
(10) Pub. No.: US 2006/0172307 A1
(43) Pub. Date: Aug. 3, 2006

(54) Method of nucleotide identification using an off-switch through proofreading 3' exonuclease-resistant modified primers by polymerases with 3' exonuclease activity

(76) Inventors: Kai Li, Glendora, CA (US); Jia Zhang, Glendora, CA (US)

Correspondence Address: RAYMOND Y. CHAN, 108 N. YNEZ AVE., SUITE 128, MONTEREY PARK, CA, 91754, US

(21) Appl. No.: 10/639,746

(22) Filed: Aug. 11, 2003

(30) Foreign Application Priority Data
Aug. 19, 2002 (CN) 02129166.7

(51) Int. Cl. C12Q 1/68; C12P 19/34

(52) U.S. Cl. 435/6; 435/91.2

(57) ABSTRACT

Methodology for a high fidelity polymerase-mediated primer extension for use in genotyping is provided. The primer extensions are carried out with polymerase having 3′ to 5′ exonuclease activity and primers with their 3′ modification of being exonuclease-resistant. The primers are designed to have their 3′ termini: 1. complementary to nucleotide to be analyzed; and 2. subject to be proofread by the 3′ to 5′ exonuclease of the high fidelity polymerases.

### CROSS REFERENCE OF RELATED APPLICATION

This patent application is partially cross-reference to a prior separate application: (USPTO disclosure 486671, dated Dec. 28, 2000) application Ser. No. 09/808,659, filed Mar. 14, 2001. It is also cross-reference to a prior Chinese patent application, application number: 02129166.7, filed Aug. 19, 2002.

### BACKGROUND OF THE PRESENT INVENTION

1. Field of Invention

The invention generally relates to single nucleotide discrimination technology. In particular, the invention provides a method that utilizes high fidelity polymerases-mediated primer extension with 3′ exonuclease-resistant modified primers.

2. Description of Related Arts

Single nucleotide polymorphisms are the most common form of genetic variation. In the postgenome era, efficient screening of known SNPs is of paramount importance as it can maximize the value of sequence data from the human genome project in critical applications such as fundamental medical research and individualized medicine. At present, the high rate of false positives is one of the major obstacles to the effective application of available high throughput SNP assays, preventing them from being more widely used in clinical applications. A high rate of false positives occurs in multiple SNP assays mainly as a result of the narrow range of thermal hybridization and wash conditions used to differentiate perfect match from single-base mismatch.

Although it has been well known for decades that polymerases with proofreading activity have higher fidelity in DNA chain elongation, it could not been used in SNP genotyping since identical products are produced from matched and mismatched allele-specific primers using conventional strategy.

We had a new algorithm of using high fidelity polymerases in SNP assay and have done a series studies to reduce the practice. In addition to the new SNP assay by 3′ terminal labeled primer extension (our previous U.S. patent application Ser. No. 09/808659, filing date on Mar. 14, 2001, ref 1-4). We recent suggested a new theory about fidelity maintenance through the off-switch by polymease (5,6,7). Based on this new ON/OF switch theory, we developed the second generation of high fidelity polymerase-mediated SNP assay using the combination of high fidelity polymerases and 3′ exonuclease-resistant primers.

### SUMMARY OF THE PRESENT INVENTION

The present invention provides primer extension design that is consisted of the 3′ exonuclease-resistant modified primers and DNA polymerase having 3′ to 5′ exonucleases function.

In one embodiment, the invention provides a method of genotyping single nucleotide or single nucleotide polymorphism in a DNA sample. The method includes the steps of:

1. conducting primer extension mediated by high fidelity polymerase with 3′ modified primers that are 3′ exonuclease-resistant; and

2. identifying extended products from primer extension. The step of identification allows to genotype specific nucleotide of target DNA.

In one embodiment of the method, the target loci is homozygo of wild type, one specific products amplified from the extension of 3′ modified primers complimentary to wild type-specific allele. Primers that do not match templates are not extended as attributed to primers′ exonuclease-resistance.

In one embodiment of the method, the target loci is homozygo of mutant type, one specific products amplified from the extension of 3′ modified primers complimentary to mutant type-specific allele. Primers that do not match templates are not extended.

In one embodiment of the method, the target loci is heterozygo of wild type and mutant type, two specific products amplified from the extension of 3′ modified primers complimentary to wild type-specific allele and from the extension of 3′ modified primers complimentary to mutant type-specific allele.

### BRIEF DESCRIPTION OF THE DRAWINGS

FIG. 1 is a schematic representation of primer extension mediated by high fidelity polymerase using 3′ modified allele-specific primers that are resistant to 3′ to 5′ exonuclease. PCR products were only extended from matched primers, whereas 3′ mismatched primers were not extended due to a premature termination of DNA polymerization through an off-switch action operated by the 3′ to 5′ exonuclease of the high fidelity polymerase.

FIG. 2 illustrates an on/off switch being observed using phosphorothioate-modified allele-specific primers and polymerase having 3′ to 5′ exonuclease function in a broad range of annealing temperature.

FIG. 3 illustrates an off-switch being observed using phosphorothioate-modified allele-specific primers with mismatched nucleotide at or near the 3′ terminal and polymerase having 3′ to 5′ exonuclease function.

FIG. 4 illustrates an human genomic DNA being directly assayed by the on/off switch consisted of phosphorothioate-modified allele-specific primers and polymerase having 3′ to 5′ exonuclease function.

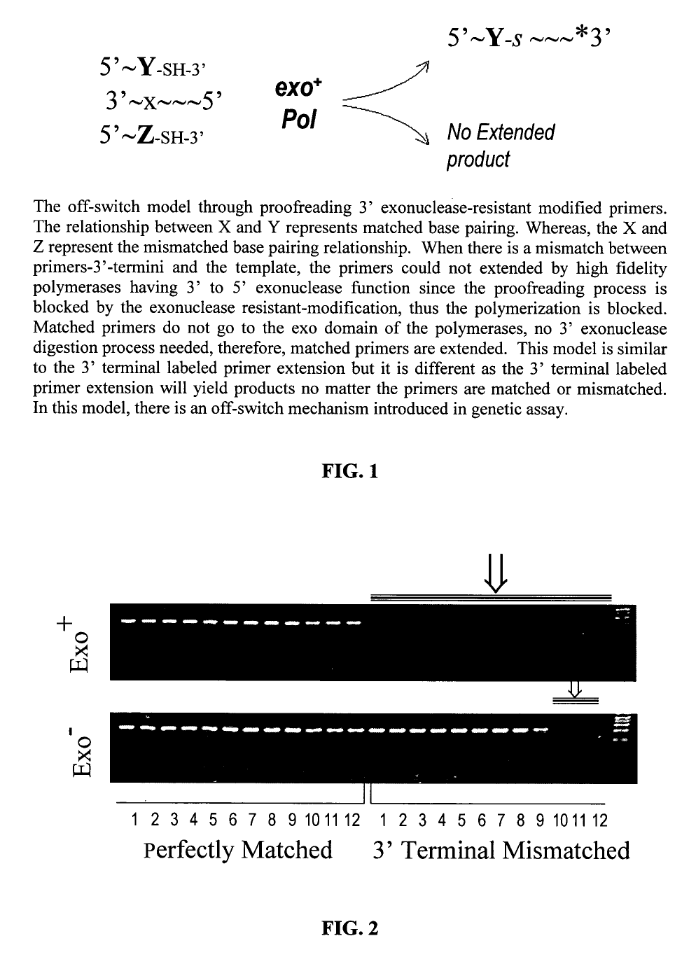

### DETAILED DESCRIPTION OF THE PREFERRED EMBODIMENT

The present invention provides a new method of primer extension for genotyping the base identification of target DNA. The new method is highly affordable, and makes use of popular and readily accessible agarose gel electrophorosis and DNA sequencers for analysis, and also adaptable for microplate and microarray technologies.

The method of the present invention introduces specific modification on 3′ terminal or near the 3′ terminal of allele-specific primers and uses the high fidelity DNA polymerase having 3′ to 5′ exonuclease function. The combination of 3′ modification of being exonuclease-resistant and the high fidelity DNA polymerase having 3′ to 5′ exonuclease activity forms a on/off switch in discrimination of single nucleotide sequences of target DNA sample, allowing perfect matched primers to extend. Whereas, mismatched primers are subjected to be proofread before they could be extended. The exonuclease-resistant property of the modification blocked the procedure of proofreading, thus leading to the failure of enzymatic excision of mismatched 3′ terminal and cause premature-termination in DNA polymerization, an off-switch effect as described in our publications in scientific journals.

The present invention thus utilizes a serial linkage of two relatively high chemical reactions to reach a higher accurate and selectivity. The first reaction is a general matching testing by polymerase domain. The second reaction is the proofreading procedure executed by the internal 3′ to 5′ exonuclease. The internal 3′ to 5′ exonuclease has a very high specificity to remove the nucleotides at 3′ terminal of mismatched primers with little matched primers digested.

The fundamentals of this design for primers are that single nucleotides are complimentarily located on primers′ 3′ end, at the 3′ terminal or near the 3′ terminal, and the exonuclease resistant modifications are added on the nucleotides complementary to the target nucleotides.

In evaluating the potential of exo+ polymerase in SNP analysis, we identified the crucial role of successfully 3′ exonuclease excision in DNA polymerization from mismatched primers using exo+ polymerase. In addition to the well-known proofreading function, 3′ exonuclease actually plays a dual role in maintaining fidelity of DNA polymerization. Successful mismatch excision is required for polymerization from mismatched primers, whereas failure in mismatch excision turns off DNA polymerization. For DNA dependent polymerase having proofreading function, whether DNA polymerization occurs mainly depends on the process of mismatch excision by 3′ exonuclease. When the mismatch is removed by 3′ exonuclease, DNA polymerization turns to the on status with the yield of template-dependent products. This proofreading function cannot by directly used in SNP genotyping. In a previous patent application, the 3′ terminal labeled primer extension, we invented a new SNP assay based on the proofreading function of the high fidelity polymerases with 3′ to 5′ exonucleases activity. On the contrary, if the mismatched nucleotide is not excised DNA polymerization stays at the off status leading to a premature termination. Premature termination worked as a way to minimize mutagenesis in matured polymerization products.

However, premature termination in DNA polymerization occurs in a very low incidnece in vivo. It either cannot directly used in SNP genotyping as most of the 3′ terminal mismatched primers would be efficiently corrected during proofreading process by the internal 3′ to 5′ exonucleases. The introduction of 3′ exonucleases-resistant modified is essential in order to applying the premature termination in SNP genotyping. One example of such modifications, is phosphorothioate-modification. Phosphorothioate modification has been known for decades to render nucleases resistance. A molecular off switch was devised with the combination of high fidelity polymerases and 3′ exonucleases-resistant modified primers in primer extension. No products were yielded from mismatched primer with 3′ phosphorothioate modification by exo+ polymerase was reasonable as the 3′ phosphorothioate-modification blocked exonuclease function.

The inefficient excision by 3′ exonuclease acts as an off-switch, which turns off DNA polymerization from mismatched primers that are resistant to 3′ exonucleases proofreading. Exo+ polymerases use this off-switch mechanism to maintain the high fidelity in DNA synthesis by truncating the products with mutations. Although the biological implication of this novel mechanism remains to be elucidated, we have successfully developed SNP assay depending on the premature termination of DNA synthesis by the off. For in vitro primer extension, this off-switch is the scientific bases for the new SNP assay. As showed in the example given in this application, a point mutation associated with deafness was discriminated as diagnostic assay: DNA polymerization was turned off from primers targeting mutated allele, encouraging the application of 3′ PTO-modified primers and exo+polymerases in SNP assay and in diagnosis for SNP-related genetic diseases.

As compared to most of the SNP assay, the on/off switch does not require prior amplification in the discrimination of SNP using genomic DNA samples. Requirement of prior amplification of genes is a bottleneck restraining the high throughput screening of known SNPs in the postgenome. The present invention has demonstrated the ability of the newly identified on/off switch in SNP analysis with genomic DNA sample directly. The advantage of using genomic DNA samples in high throughput screening of a large number of SNPs is enormous. Prior amplification before single base discrimination is inconvenient and laborious but still possible for single or a limited amount of SNPs, whereas it is technically difficult for high throughput screening of hundreds to thousands different types of SNPs, either with microarray or microtiter technologies.

Those of skill in the art will recognize that the primer extension condition may vary depending on platforms employed. 5 Electrophoresis and visualization: This category covers broad range of electrophoresis applications and a variety of visualization methods. As long as the sizes, the colors, and the intensities of signal of the products are not exactly the same, visualization is able to differentiate the different alleles of the SNP to be analyzed.

Those of skill in the art will recognize that ways to analyze the data obtained from a primer extension varied from the platforms of technologies applied to. For example, agarose gel electrophorosis shows results by the presence or absence of primer-extended products. The real time PCR shows signal curves expressed by cycles of threshold (Ct). For the case of microarray or microplate, the color or the ratio of intensities of the reporting dyes is used as the parameters for genotyping.

### EXAMPLES

### Example 1

The 3′ phosphorothioate-modified allele-specific primer together with exo+ polymerase formed an on/off switch for SNP discrimination. These data will be published in Laboratory Investigation, Molecular Biotechnology, Journal of Biochemistry and Molecular Biology, and Trends in Biotechnology in 2003 and 2004.

The amplicon set used in this study was from Genomapping Inc. (Tianjin, China), which included two templates differed from each other at a single nucleotide, two sense primers, and one antisense primer. The two templates of this amplicon had the following sequences with the polymorphism underlined and bolded: 5′-atcccaagatatctgagaatt(c/g)tcagcagccttccatagaagggtgttgttgtctctgaggcaaaaccacatttcttaccgca caactagagactgagaccagtctctcattgtcattgctgctcagagccagcagaaaagcactcatgacacacacttagaataat agtgcatctgagccaggactgcccttggggtccattcagctgtttc-3′. The two sense primers had the identical sequence of 5′-atcccaagatatctgagaattc-3′. One of the sense primers was unmodified and another sense primer had 3′ terminal phosphorothioate modification. The antisense primer had the sequences of 5′-cagtctctagttgtgcggtaagaaat-3′ without phosphorothioate modification.

Two types of DNA-dependent DNA polymerases were purchased from New England Biolab Inc. (Beverly, Mass., USA), Deep vent and Deep Vent-. Deep Vent is the wild form that contains a strong 3′ to 5′ exonuclease activity, which was evaluated for its potential in SNP analysis. Deep Vent- is the form with a point mutation that resulted in the loss of proofreading function.

Gradient two-directional primer extensions were carried out with annealing temperature ranging from 46 to 66° C. The primer extension with matched amplicon employed the template harboring the sequence of 3′-cttaag-5′ and the template harboring the sequence of 3′-cttaac-5′was used in primer extension with single base mismatched amplicon (Table 1). Following denature at 95° C. for two minutes, primer extension was cycled with 30 seconds denture at 95° C., 30 seconds annealing, and 30 seconds extension at 72° C. for 30 cycles. After the 30 cycles, an extra extension cycle with 2 minutes was done before the reactions were cooled down to 4° C. The primer extension reaction was performed in a total volume of 20 μl with 10 pg of template, 0.2 mM dNTP, 0.01 μ/μl of polymerase, 10 pmol/μl of both sense and antisense primers, and 2 μl of the 10× NEB polymerase reaction buffer which provides a final concentration of 10 mM KCl, 20 mM Tris-HCl (pH 8.8 at 25° C.), 10 mM (NH4)2SO4, 2 mM MgSO4, 0.1% Triton X-100. PCR products were visualized using 2% agarose EtBr gel electrophoresis running under 10 volt/cm in TBE.

Mismatch proofreading turned off DNA polymerization from 3′ phosphorothioate-modified primers. With amplicon containing a single base mismatch between 3′ termini of the 3′ phosphorothioate-modified primers and the templates (template harboring the sequence of 3′-cttaac-5′), DNA polymerases with or without proofreading function had very different effect on primer extension. As a control, DeepVent-, DNA polymerase lacking proofreading function, efficiently yielded primer-extended products from 3′ mismatched primers at annealing temperature at 62.8° C. or lower. This was similar to our previous observation using unmodified primers amplified by exo-polymerase. When applied to practical SNP assay, the polymerization from mismatched primers might be the major source of false-positives.

A breakthrough phenomenon was observed when proofreading phosphorothioate-modified primer-3′-termini occurred in the case of a mismatch between primer-3′-termini and the templates. The primers with phosphorothioate-modified 3′ termini were not extended at any annealing temperature within the range tested when there was a single base mismatch between the primer-3′-termini and the templates (FIG. 2). With the matched amplicon, 3′ terminal phosphorothioate-modified primers were well extended as no proofreading was processed. These data illustrated a perfect on/off switch in DNA polymerization in primer extension with exo+ polymerase having proofreading function (Table 1): DNA polymerization turned on when matched and turned off when there was a single base mismatch between phosphorothioate-modified primer-3′-termini and the templates.

### Example 2

The 3′ phosphorothioate-modified primers used for nucleotide identification upstream of primer-3′-terminal.

This study was designed to determine the base identification ability of high fidelity polymerases on primers with mismatches upstream from the 3′ phosphorothioate-modified terminus. Therefore, this example was a further expand of the on/of switch in genetic analysis.

Unmodified primers were synthesized commercially by Sengon Inc, (Shanghai, China) and phosphorothioate-modified primers were synthesized by MWG biotech AG (Charlotte, N.C., USA). Deep Vent- and Deep Vent were purchased from New England Biolab Inc. (Beverly, Mass., USA). Deep Vent is a wild type polymerase having 3′ to 5′ exonuclease activity, and Deep Vent- is the mutant form after point mutation that eliminated the internal 3′ to 5′ exonuclease function.

Two-directional primer extensions were set at annealing temperature of 56° C. Following denature at 95° C for two minutes, primer extension was cycled with 30 seconds denture at 95° C., 30 seconds annealing, and 30 seconds extension at 72° C. for 30 cycles. After the 30 cycles, an extra extension cycle with 2 minutes was done before the reactions were cooling down to 4° C. The primer extension reaction was performed in a total volume of 40 μl with 20 pg of template, 0.2 mM dNTP, 0.01 u/μl of polymerase, 10 pmol/μl of both sense and antisense primers, and 4 μl of the 10× NEB polymerase reaction buffer which provides a final concentration of 10 mM KCl, 20 mM Tris-HCl (pH 8.8 at 25° C.), 10 mM (NH4)2SO4, 2 mM MgSO4, 0.1% Triton X-100.

A set of seven templates differing from each other by single nucleotide is used (difference is capitalized and underlined below):

Template 1:
5′-atcccaagatatctgaGAATTCtcagcagccttccatttagaagggt
gttgttgtctctgaggcaaaaccacatttcttaccgcacaactagagact
gagaccagtttctctcattgtcattgctgctcagagccagcagaaaagca
ctcatgacacacacttagaataatagtgcatctgagccaggactgccctt
ggggtccattcagctgtttc-3′;
Template 2:
5′atcccaagatatctgaGAATTG tcagcagc . . . tc3′;
Template 3:
5′atcccaagatatctgaGAATAC tcagcagc . . . tc3′;
Template 4:
5′atcccaagatatctgaGAAATC tcagcagc . . . tc3′;
Template 5:
5′atcccaagatatctgaGATTTC tcagcagc . . . tc3′;
Template 6:
5′atcccaagatatctgaGTATTC tcagcagc . . . tc3′;
Template 7:
5′atcccaagatatctgaCAATTC tcagcagc . . . tc3′.

Both polymerases, Deep Vent and Deep Vent- were employed in two-directional primer extensions using the seven short templates with a 3′ phosphorothioate-modified (as underlined) sense primer of TCCCAAGATATCTGAGAATTC and an antisense primer of CAGTCTCTAGTTGTGCGGTAAGAAAT. PCR was performed under the condition as described in the section of Materials and Methods. PCR products were analyzed with a 2% agarose gel electrophoresis. As we have demonstrated that polymerases lacking 3′ to 5′ exonuclease activity can extend 3′ mismatched primers, Deep Vent- was then used as a control to monitor the off action. The positive control of a template with a sequence of EcoR I site was served for two purposes: to monitor if the PCR reaction worked well in the selected condition and to quantitatively evaluate the efficiency of the off-action whenever there was a partial off-action.

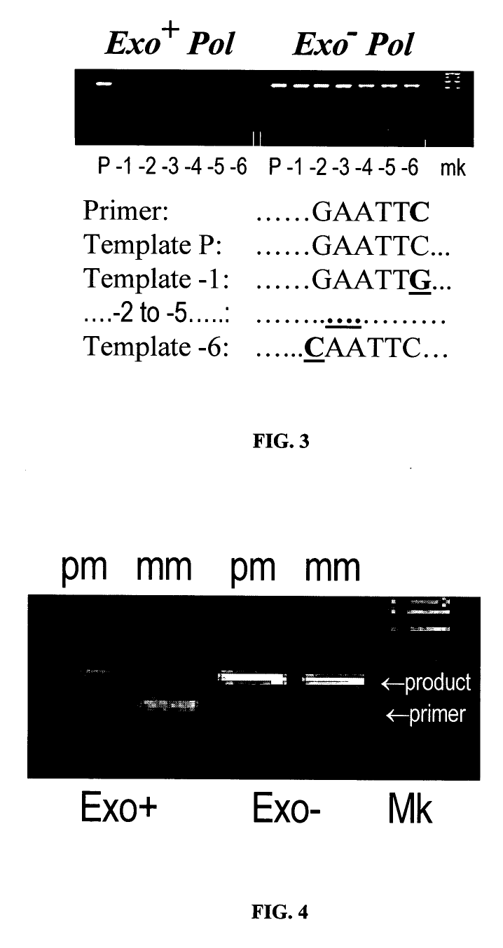

On/off switch turned off by single base mismatches upstream from primer-3-termini: With the set of seven amplicons, highly discrimination to single base mismatch upstream to the −6 position from the 3′ phosphorothioate-terminus of the primer was demonstrated using the new on/off switch (FIG. 3). DNA polymerase with proofreading activity yielded products only from perfectly matched primers, whereas DNA polymerization was halted to the six amplicons when there is a single base mismatch between templates and the 3′phosphorothioate-modified primer. This striking discrimination ability from proofreading the 3′ terminal phosphorothioate-modified primers is a valuable single nucleotide detector in SNP assay.

### Example 3

Nucleotide identification directly using genomic DNA by the combination of high fidelity polymerases and 3′ phosphorothioate-modified primers.

The human genomic DNA samples were phenol-chloroform extracted from 3 ml blood of two healthy volunteers. Primers targeting a C to T point mutation at GJB3 deafness gene were designed with 3′ phosphorothioate-modification to render exonuclease resistance. The sequence for the sense primer for C allele: 5′caa cat cgt gga ctg eta cat tgc cc3′; the antisense primer: 5′gtg aag att ttc ttc ttg gta ggt cg3′. The sense primer for the point mutated allele T: 5′caa cat cgt gga ctg cta cat tgc ct; the antisense: 5′gtg aag att ttc ttc ttg gta ggt ca3′. Two directional primer extension was performed with the condition identical to those used in example 2.

As shown in FIG. 4, the novel on/off switch, showed the ability to identify nucleotide of the locus of the recently identified GJB3 deafness gene directly using genomic DNA sample. Two allele-specific primers, sense primer and antisense primer, were used targeting the wild or mutant allele of GJB3 deafness gene. Using the genomic DNA from normal volunteers and exo+polymerase, while the primers for normal allele were extended, no products were amplified from the 3′ phosphorothioate modified l0 primers targeting C to T point mutation at GJB3 deafness gene. Similar to the results with unmodified primers, exo-polymerase generated primer-dependent products from two types of the 3′ phosphorothioate-modified allele specific primers regardless the homozygous templates from healthy volunteers were used. As shown in FIG. 4, polymerase without 3′ exonuclease activities failed to discriminate the template at single base level in this case.

Definition:

High fidelity polymerases: Polymerases that have 3′ to 5′ exonucleases, such as Pfu, Vent, Deep Vent, Tli, and T4 DNA polymerases.

The 3′ Exonuclease-resistant modified primers: Primers with modifications that render the primers resistant to the enzymatic digestion by internal 3′ exonucleases of the high fidelity polymerases, such as the modification of phosphorothioate-modification and locked nucleic acid modification.

Primer extension: Primers are prolonged by enzymatic process using polymerases. Materials can be used for primer extension including both dNTP and ddNTP. Both unlabeled and labeled dNTP and ddNTP can be used in primer extension.

What is claimed is:

1. A method of identifying the base of a target polynucleotide comprising the steps of:
(a) reacting said target polynucleotide with a primer oligonucleotide, said primer oligonucleotide having an annealing ability to said target nucleotide; and
(b) extending said primer across said target nucleotide by high fidelity polymerases.

2. The method of claim 1 wherein said polymerases contain 3′ to 5′ exonuclease activity or said proofreading function.

3. The method of claim 1 wherein said exonuclease-resistant modification on primers comprises modification that renders 3′ to 5′ exonucleases-resistatnce, including phosphorothioate modification.


SEQUENCE LISTING (5 sequences)

SEQ ID NO: 1 - DNA, 218 bp, Artificial Sequence
  Synthetic Construct
atcccaagatatctgagaattcgtcagcagccttccatttagaagggtgttgttgtctctgaggcaaaaccacatttcttaccgcacaactagagactgagaccagtttctctcattgtcattgctgctcagagccagcagaaaagcactcatgacacacacttagaataatagtgcatctgagccaggactgcccttggggtccattcagctgtttc

SEQ ID NO: 2 - DNA, 22 bp, Artificial Sequence
  Synthetic Construct
gaaacagctgaatggaccccaa

SEQ ID NO: 3 - DNA, 20 bp, Artificial Sequence
  Synthetic Construct
tcccaagatatctgagaatt

SEQ ID NO: 4 - DNA, 21 bp, Artificial Sequence
  Synthetic Construct
tcccaagatatctgagaattc

SEQ ID NO: 5 - DNA, 21 bp, Artificial Sequence
  Synthetic Construct
tcccaagatatctgagaattc